(19) United States
(12) Plant Patent Application Publication
(10) Pub. No.: US 2002/0073456 P1
(43) Pub. Date: Jun. 13, 2002

(54) Variety of angelonia plant named 'Anstern'

(76) Inventor: Andrea Michalik, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 09/733,159

(22) Filed: Dec. 8, 2000

(51) Int. Cl. A01H 5/00

(52) U.S. Cl. PLT/263

(57) ABSTRACT

A new and distinct angelonia plant with blue two-colored flowers above medium green foliage. The plant flower is suitable for pot planting or as a bedding plant.

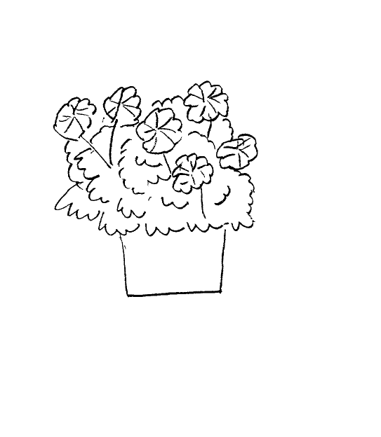

### BACKGROUND OF THE INVENTION

The present invention comprises a new and distinct cultivar of Angelonia known by the varietal name ‘Anstem’. The new variety was discovered in 1999 in a selective breeding program in Dresden, Germany. The new variety is the result of a combination breeding of 50 seedlings having blue colored flowers and a mutation of seedling An 27. The new variety exhibits the flower color of its heritage, but differs in its growth and flower shape. The new variety was first asexually reproduced by cuttings in Dresden, Germany. The new variety has been trial and field tested and has been found to retain its distinctive characteristics and remain true to type through successive propagations.

### DESCRIPTION OF THE DRAWINGS

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany. The color readings were taken on plants grown in pots outdoors in shadow during summer. Color references are primarily to The R.H.S. Colour Chart of The Royal Horticultural Society of London.

Plant:

Classification.—Botanical: Angelonia HUMB. et. BONPL.. Commercial: Angelonia.

Form.—Erect.

Height media surface to top of foliage.—50-60 cm.

Height media surface to top of cluster.—50-60 cm.

Growth.—Erect.

Strength.—Does not require artificial support.

Response time.—3 weeks.

Leaves.—Size: 10-14 cm long; 1.5-2.0 cm wide. Shape: Lanceolate. Margin: Toothed. Color: Green Group 138A. Ribs and veins: Venation: Net-shaped, not parallel. Color: Brighter than leaf surface.

Petioles.—Absent.

Stem/branches.—Color: Green Group 138A. Internode length: Vegetative: 3-4 cm. Generative: 1-1.5 cm.

The bud:

Shape when just showing color.—Overall: Racemose. Individual bud: Spherical.

Color.—Green Group 136B.

Size when just showing color.—Cluster: 5-6 cm across. Individual bud: 0.3-0.4 cm.

Inflorescence:

Blooming habit.—Single.

Size of cluster.—5-6 cm across.

Borne.—4 per nodium.

Flowers.—Form: Zygomorph. Number of flowers per cluster: 70-100. Size: 3.0-3.5 cm wide; 4.0-4.5 cm long; maximum 1 cm deep. Petals: Color: Upper surface: Violet Group 86B shading to White Group 155C at edges. Lower surface: Violet Group 86B shading to White Group 155C at edges. Size: 2.5-2.7 cm. Number per flower: 5 Shape: Ondulated. Margin: Smooth. Texture and appearance: Velvety.

Petaloids.—Absent.

Pedicel.—Length: 2.5-3 cm. Color: Medium green.

Disease/pest resistance.—No unusual susceptibility to diseases or pests has been noted to date.

Lasting quality.—Several weeks.

Reproductive organs:

Stamens.—Anthers: 0.2-0.4 cm long. Filaments: Length: 0.2-0.4 cm. Color: White. Pollen: Gray.

Pistils.—Number: 7. Length: 0.2 cm. Stigma: White. Style: 1 cm long; white.

Fruit.—Capsula.

### GENERAL CHARACTERISTICS

1. Suitable as a potting plant and as a bedding plant.

2. Possible as a cut flower.

3. Long-lasting.

4. Not susceptible to flower shattering.

5. Growth regulators not required.

6. Branching is better after pinching.

7. Two-colored flower.

8. Very decorative.

I claim:

1. A new and distinct variety of angelonia plant substantially as shown and described.
